(19) United States
(12) Plant Patent Application Publication
(10) Pub. No.: US 2002/0124288 P1
(43) Pub. Date: Sep. 5, 2002

(54) PEACH TREE NAMED 'SUPECHFIFTEEN'

(76) Inventors: Bruce D. Mowrey, La Selva Beach, CA (US); David W. Cain, Bakersfield, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/792,530

(22) Filed: Feb. 23, 2001

(51) Int. Cl. A01H 5/00

(52) U.S. Cl. PLT/197

(57) ABSTRACT

A new and distinct peach variety with large, firmly textured fruit, yellow flesh, and 50-70% red overcolor. The new variety ripens early in the season and has lower winter chilling requirements.

### BACKGROUND AND SUMMARY OF THE INVENTION

This invention relates to the discovery and asexual propagation of a new variety of peach, Prunus persica cv. ‘Supechfifteen’. The new variety was first hybridized by Bruce D. Mowery on Apr. 25, 1994, and selected and evaluated by Bruce D. Mowrey and David W. Cain near Mecca, Riverside County, Calif. The variety was originated by hybridization. The new variety cv. ‘Supechfifteen’ is characterized by possessing large, yellow-fleshed fruits that are blocky in shape with a less pronounced tip. The fruits are firm in texture, and a red overcolor is present on approximately 50-70% of the fruit surface.

The seed parent is the ‘Flordaglo’ variety (unpatented), while the pollen parent is the ‘Flordaglobe’ variety (unpatented). The parent varieties were first crossed in February 1992, with the date of sowing being October 1992. The first flowering was January 1994. The new ‘Supechfifteen’ variety was first asexually propagated by Bruce D. Mowrey in June 1994.

The new variety cv. ‘Supechfifteen’ ripens at approximately the same time as its pollen parent, the ‘Flordaglobe’ variety, but differs in several characteristics. ‘Supechfifteen’ has larger fruit (169 g compared with 115 g for ‘Flordaglobe’). The new variety also has less overcolor than ‘Flordaglobe’, (50-70% compared to 60-80% with ‘Flordaglobe’). The new variety ‘Supechfifteen’ has a lower chilling requirement than ‘Flordaglobe’, requiring approximately 150 hours of temperatures below 45° F. compared to 500 hours for ‘Flordaglobe’. The new variety ‘Supechfifteen’ may be distinguished from its seed parent, ‘Flordaglo’ by having yellow flesh as compared to the white flesh of ‘Flordaglo’.

The new variety cv. ‘Supechfifteen’ may be distinguished from presently available low-chilling varieties in commerce by several characteristics. ‘Supechfifteen’ ripens approximately three days before ‘Flordaprince’, and has larger-sized fruit than ‘Flordaprince’ (approximately 169 g per fruit compared to 110 g per fruit in the ‘Flordaprince’ variety). The new variety ripens approximately 13 days before the ‘Tropic Beauty’ variety.

The new peach variety cv. ‘Supechfifteen’ has been shown to maintain its distinguishing characteristics through successive asexual propagations by, for example, budding.

### BRIEF DESCRIPTION OF THE FIGURE

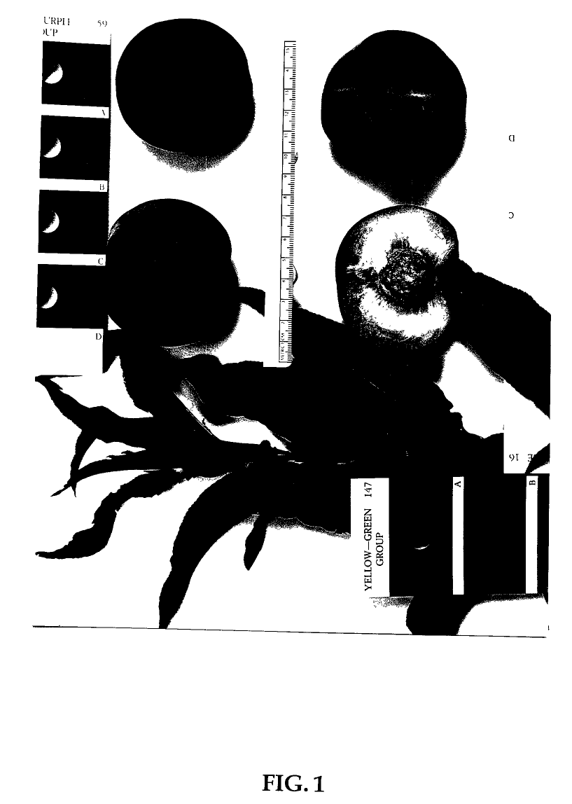

The accompanying drawing in FIG. 1 illustrates in full color.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon the R.H.S. Colour Chart, published by The Royal Horticultural Society, London, England.

The descriptive matter which follows pertains to ‘Supechfifteen’ plants grown in the vicinity near Wasco, Kern County, Calif., during 2000, with bloom and ripe dates also given for a low chill site near Mecca, Riverside County, California. The descriptive matter is believed to apply to plants of the same variety grown under similar conditions of soil and climate elsewhere:

### TREE

General: Trees were five years old and were maintained at a height of about ten feet and a canopy diameter of about ten feet by annual prunings.

Size.—medium.

Vigor.—medium.

Habit.—semi-upright.

Density of Foliage.—medium.

Shape.—vase formed.

Hardiness.—hardy near Wasco in Kern County, Calif.

Productivity.—productive.

Fruit bearing.—regular bearer.

Root stock.—Nemared.

Trunk: Trunk is round in outline and of medium circumference, averaging 68 cm at 30 cm above ground level on 5-year-old trees.

Shape.—medium.

Surface texture.—smooth-medium.

Color.—about 199B Grey-Brown group.

Branches: Branches are round in outline and average 37 cm in circumference on 5-year-old trees as measured 30 cm distal from the branch juncture with the trunk.

Shape.—medium-slender.

Surface texture.—smooth-medium.

Surface appearance.—semi-glossy-dull.

Lenticels:

Number.—medium-few.

Size.—large-medium.

Density.—1.6/cm².

### LEAVES

General:

Average length.—about 18 cm.

Average Width.—about 4.9 cm.

Size.—large-medium.

Outline.—lanceolate.

Profile.—up folded.

Leaf blade tip.—curved downwardly.

Angle of tip.—acute.

Margin.—finely serrate

Undulation of margin.—slight.

Apex.—acuminate.

Base.—U-shaped.

Thickness.—medium.

Upper Surface:

Color.—about 147A Yellow-Green group.

Glossiness.—medium.

Pubescense.—absent.

Surface texture.—rugose.

Surface appearance.—semi-glossy.

Lower Surface:

Color.—about 147B Yellow-Green group.

Glossiness.—weak.

Pubescense.—absent.

Surface texture.—rugose.

Surface appearance.—dull.

Petiole:

Length.—medium-short, about 1 cm.

Thickness.—thick-medium, about 2 mm.

Glands:

Average number.—5.

Positioning.—on both leaf base and petiole.

Size.—medium.

Shape.—reniform.

Color.—about 153D Yellow-Green group.

Stipules:

Persistence.—fall off.

Wood (Leaf) Buds:

Shape.—ovoid.

Size.—small-medium.

Position relative to shoot.—adpressed.

Support.—decurrent.

Time of bud burst.—medium.

### FLOWERING SHOOTS:

Anthocyanin coloration.—present.

Intensity.—medium.

Thickness.—thin-medium, about 0.40 cm.

Internode length.—medium, about 2.3 cm, midway on flowering shoots.

Density of buds.—medium.

Distribution.—on one year old shoots.

Ratio of wood (leaf) buds to flowering shoots.—1:2.5.

### FLOWERS

Flower-Buds:

Hardiness.—hardy under California conditions.

Size.—medium.

Length.—medium.

Shape.—pointed—plump.

Positioning.—free.

Pubescence. pubescent.

Color.—about 165A Greyed-Orange group.

General:

Date of first bloom at low chill site.—Jan. 24, 2000, near Mecca, Riverside County, Calif.

Date of full bloom at low chill site.—Jan. 29, 2000, near Mecca, Riverside County, Calif.

Date of first bloom at medium-high chill site.—Feb. 1, 2000, near Wasco, Kern County, Calif.

Date of full bloom at medium-high chill site.—Feb. 6, 2000, near Wasco, Kern County, Calif.

Time of bloom.—medium, as compared with similar varieties in the growing area of Mecca, Riverside County, Calif.

Duration of bloom.—medium, about 10 days.

Size (diameter of the fully open flower).—large, about 4.8 cm.

Shape.—rosaceous.

Petals.—overlapping.

Color.(fully opened flower).—about 62D.

Peduncle:

Length.—short.

Thickness.—medium.

Pubescence.—absent.

Receptacle:

Depth.—medium.

Pubescence of inner surface (at white bud stage).—absent.

Pubescence of outer surface.—absent.

Sepals:

Positioning.—adpressed to petals.

Shape.—ovate.

Pubescence of inner surface.—absent.

Pubescence of outer surface.—medium.

Frequency of flowers with double sepals.—none.

Petals:

Frequency of flowers with double petals.—none.

Size.—medium, about 1.6 cm width.

Shape.—transverse broad elliptic.

Claw length.—medium.

Margin waviness.—medium.

Base angle.—medium.

Division of upper margin.—entire.

Pubescence of inner surface.—absent.

Pubescence of outer surface.—absent.

Color (inner surface).—about 62D.

Color (outer surface).—about 62D.

Stigma:

Position (as compared with anthers).—level.

Anthers:

Color (just before dehiscence).—about Red 46D.

Pollen:

Color .—about 14B.

Stamens:

Position.—perigynous.

Pistil:

Frequency of supplementary pistils.—few.

Number.—sometimes more than one.

Ovary:

Pubescence.—pubescent.

Density of hairs.—medium.

Style:

Pubescence (of base).—medium.

Density of hairs.—slight.

### FRUIT

General:

Maturity when described.—firm-ripe.

Date.—Apr. 12, 2000 at the low-chill site near Mecca, Riverside County, Calif. May 6, 2000 at the medium-high chill site near Wasco, Kern County, Calif.

Size.—slightly variable; large, about 7.3 cm, about 169 g.

Axial Diameter.—about 6.8 cm.

Transverse in suture plane.—about 7.3 cm.

At right angles to suture plane.—about 7.3 cm.

Form.—slightly variable; globose.

Position of maximum diameter.—towards middle.

Symmetry about the suture.—slightly asymmetric.

Season ripening.—early.

Use.—fresh market.

Keeping quality.—medium.

Resistance to.—Insects: good. Diseases: good.

Shipping quality.—medium.

Suture: an inconspicuous line; has slight-marked depression beyond pistil point.

Ventral Surface:

Shape.—rounded slightly.

Lips.—equal.

Depression of apex.—distinct.

Pistil base.—not persisting.

Pubescence at apex.—medium.

Density of apex pubescence.—medium.

Stem Cavity:

Shape.—flaring; circular; with suture showing on one side.

Depth.—about 1.8 cm.

Breadth.—about 3.0 cm.

Base:

Shape.—rounded.

Apex:

Shape.—slightly depressed.

Pistil Point:

Shape.—slightly depressed.

Stem:

Length.—about 0.8 cm.

Appearance.—stout, glabrous.

Adherence to stone.—strong.

Skin:

Thickness.—medium.

Texture.—medium.

Reticulation.—absent.

Roughness.—absent.

Tenacity.—tenacious to flesh.

Tendency to crack.—none in wet season.

Ground color.—about 1C.

Overcolor.—about 59A.

Down (pubescence).—moderate to scant.

Flesh:

Color.—about Green-Yellow group 1B near stem end; about Red-Purple group 59B in flesh near apex.

Amygdalin.—wanting.

Juice.—moderate.

Sugar content.—low to medium.

Texture.—medium, coarse, melting.

Fibres.—abundant, coarse, tender.

Ripens.—earliest at apex.

Flavor.—delicate.

Eating quality.—good for early season fruit.

Stone/flesh ratio.—1:3.

### STONE

General:

Adherence to flesh.—cling—adheres to flesh over entire surface.

Fibres.—long.

Size.—medium.

Length.—about 3.2 cm.

Width along suture plane.—2.2 cm.

Width at right angle to suture plane.—1.8 cm.

Width of stalk end.—about 0.4 cm.

Angle of stalk end.—obtuse.

Thickness.—about 1.2 cm.

Form (profile).—oval.

Form (ventral view).—globular.

Base.—oblique.

Hilum.—oval.

Apex.—pointed.

Position of maximum breadth.—middle.

Sides.—equal.

Surface.—regularly furrowed near base, pitted throughout.

Outgrowing keel.—partly developed.

Ridges.—rounded towards base.

Pits.—elongated.

Ventrical edge.—thin with slight wing toward base.

Dorsal edge.—narrow.

Color of stone.—about 165D Greyed-Orange group.

Tendency to split.—slight in wet season.

What is claimed is:

1. A new and distinct variety of peach cv. ‘Supechfifteen’ as herein illustrated and described.
